(19) United States
(12) Patent Application Publication
(10) Pub. No.: US 2026/0191876 A1
(43) Pub. Date: Jul. 9, 2026

(54) TREATMENT OF HEAD AND NECK SQUAMOUS CELL CARCINOMA

(71) Applicant: OHIO STATE INNOVATION FOUNDATION, Columbus, OH (US)

(72) Inventor: Steve OGHUMU, Columbus, OH (US)

(21) Appl. No.: 19/431,184

(22) Filed: Dec. 23, 2025

Related U.S. Application Data
(60) Provisional application No. 63/741,626, filed on Jan. 3, 2025.

(51) Int. Cl. A61K 31/5377; A61K 45/06; A61P 35/00

(52) U.S. Cl. CPC A61K 31/5377; A61K 45/06; A61P 35/00

(57) ABSTRACT

Disclosed herein are compositions and methods for treating head and neck squamous cell carcinoma.

### CROSS-REFERENCE TO RELATED APPLICATIONS

This application claims the benefit of priority to U.S. Provisional Patent Application No. 63/741,626, filed Jan. 3, 2025, the disclosure of which is incorporated herein by reference in its entirety.

### BACKGROUND

Head and Neck Squamous Cell Carcinoma (HNSCC) is the 7th most common cancer worldwide, with a 5-year survival rate of 40-50%. Human Papilloma Virus (HPV) negative HNSCC, partly attributable to alcohol and tobacco consumption, is associated with a worse prognosis. Treatment for HPV negative HNSCC consists of radiation and highly invasive surgery, which causes disfigurement. Chemoradiotherapy, with cisplatin/docetaxel followed by radiation, has increased progression-free survival in recent clinical studies. However, chemoradiotherapy-related toxicities have become a growing concern with neutropenia, thrombocytopenia, and dysphagia, ruling out elderly patients and those with comorbidities due to treatment-related deaths. Recently, molecular and immune therapies have emerged as viable treatment options, with multi-modal approaches including anti-PD-1 antibody, VEGF and mTOR inhibitors, and EGFR inhibitors such as cetuximab demonstrating modest increases in median overall survival when combined with chemotherapy. Although PD-1 checkpoint blockade has been approved as a first-line treatment for advanced HNSCC patients, underlying mechanisms of enhanced treatment responses, as well as optimal therapeutic regimens for improved disease and progression-free survival, are still incompletely understood.

There remains a need for improved compositions and methods for treating head and neck squamous cell carcinoma. There remains a need for improved compositions and methods for targeting the HNSCC tumor microenvironment to enhance antitumoral efficacy.

### BRIEF DESCRIPTION OF THE FIGURES

FIGS. 1A-1F depict and provide data regarding: (FIG. 1A) Tumor volumes of MOC-2 and MOC-1 injected mice treated with CPSI-1306 or vehicle control, measured in mm³; (FIG. 1B) Tumor sizes of tumors from vehicle control and CPSI-1306 treated MOC-2 and MOC-1 injected mouse groups, as determined by image J analysis; (FIG. 1C) Images of tumors from MOC-2 injected mice treated with vehicle control and CPSI-1306; (FIG. 1D) Images of tumors from MOC-1 injected mice treated with vehicle control and CPSI-1306; (FIG. 1E) Representative immunohisto-chemistry (IHC) images of tumors of mice injected with MOC-2 cancer cells stained with MIF antibody; (FIG. 1F) Representative IHC images of spleen tissue of wild type C57BL/6 or Mif knock-out mice stained with MIF antibody. Data reported as mean±SEM, n>=5, *P<0.05.

FIGS. 2A-2F depict and provide data regarding: (FIG. 2A) Flow cytometry gating strategy of T cell from single cell suspensions derived from tumors of mice treated with CPSI-1306 or vehicle control. Gating for CD4⁺ and CD8⁺ cells was performed on CD45⁺CD3⁺ cells; (FIG. 2B) Representative flow cytometry plot for CD4⁺ and CD8⁺ T cells in MOC-2 tumors of mice treated with vehicle control and CPSI-1306; (FIG. 2C) Graphical representation of CD4⁺ and CD8⁺ T cell subsets expressed as a percentage of CD45⁺ cells and total live cells in MOC-2 tumors of CPSI-1306 and vehicle control treated groups; (FIG. 2D) Representative flow cytometry plot for CD4⁺ and CD8⁺ T cells in MOC-1 tumors of mice treated with vehicle control and CPSI-1306; (FIG. 2E) Graphical representation of CD4⁺ and CD8⁺ T cell subsets expressed as a percentage of CD45⁺ cells and total live cells in MOC-1 tumors of CPSI-1306 and vehicle control treated groups; (FIG. 2F) Gene expression profile of chemokines Ccl5, Cxcl9, Cxcl10, Cxcl11, and Ccl21 in tumors obtained from tumor bearing mice treated with CPSI-1306 or vehicle control, normalized to β-actin. *p value <0.05, **p value <0.01.

FIGS. 3A-3L provide representative flow cytometry plots and summary bar graphs showing percentage of CD4⁺ and CD8⁺ T cells, respectively, expressing (FIGS. 3A, 3B, and 3C) CTLA-4, (FIGS. 3D, 3E, and 3F) TIM3, (FIGS. 3G, 3H, and 3I) TIGIT and (FIGS. 3J, 3K, and 3L) PD-1 in spleens (SP), draining lymph nodes (LN), and tumors (TM) of MOC-2 and MOC-1 injected mice treated with a vehicle control or CPSI-1306. Data are presented as mean±SE. ***P value <0.001, **P value <0.01, *P value <0.05.

FIGS. 4A-4D provides representative flow cytometry plots with associated summary bar graphs showing percentage of CD4⁺ and CD8⁺ T cells expressing (FIG. 4A) IFN-γ, (FIG. 4B) TNF-α and (FIG. 4C) Gzmb in Lymph nodes and Spleens of MOC-2 and MOC-1 injected tumor bearing mice treated with vehicle control or CPSI-1306. (FIG. 4D) Representative immuno-fluorescence staining of primary tumor tissue from vehicle control and CPSI-1306 treated MOC-2 tumor bearing mice stained with DAPI, CD8, and Gzmb. Composite image is also shown to indicate CD8 cells expressing Gzmb. Bar graph depicting number of Gzmb producing CD8⁺ cells in tumors of vehicle control or CPSI-1306 treated mice. Quantification of graphical images were taken from four fields with N=5 animals per group. Data are presented as mean±SE. **P value <0.01, *P value <0.05.

FIGS. 5A-5J depict and provide data regarding: (FIGS. 5A-5E) Histogram plots of (FIG. 5A) human oral epithelial cell line TE1177, (FIG. 5B) human cancer cell lines SCC83, (FIG. 5C) CA83, (FIG. 5D) CAL27, and (FIG. 5E) mouse oral cancer cell line MOC-2, stained with MIF antibody or IgG isotype control antibody, and analyzed by flow cytometry; (FIGS. 5F-5J) Western blot images and graphical quantification of pERK, pAKT, and total ERK protein expression in (FIG. 5F) TE1177 normal oral epithelial cell line, (FIG. 5G) SCC83, (FIG. 5H) CA83, (FIG. 5I) CAL 27 and (FIG. 5J) MOC-2 HNSCC cell lines treated with vehicle or CPSI-1306 for 24 h at 1 and 10 μM. Western blot images were quantified with Image J, with proteins were normalized against GAPDH. *p value <0.05; **p value <0.01; ***p value <0.001.

### DETAILED DESCRIPTION

Before the present methods and systems are disclosed and described, it is to be understood that the methods and systems are not limited to specific synthetic methods, specific components, or particular compositions. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” mean “including but not limited to,” and is not intended to exclude, for example, other additives, components, integers, or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed, that while specific reference of each various individual and collective combinations and permutations of these may not be explicitly disclosed, each is specifically contemplated and described herein, for all methods and systems. This applies to all aspects of this application, including, but not limited to, steps in disclosed methods. Thus, if there are a variety of additional steps that can be performed, it is understood that each of these additional steps can be performed with any specific embodiment or combination of embodiments of the disclosed methods.

Compounds disclosed herein may be provided in the form of acceptable salts, for example, pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The term “alkyl” refers to a radical of a straight-chain or branched hydrocarbon group having a specified range of carbon atoms (e.g., a “C₁₋₁₆ alkyl” can have from 1 to 16 carbon atoms). An alkyl group can be a saturated alkyl group or an unsaturated alkyl group, i.e., an alkyl group having one or more carbon-carbon double/triple bonds, i.e., an alkenyl or alkynyl group. Unless specified to the contrary, an “alkyl” group includes both saturated alkyl groups and unsaturated alkyl groups.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

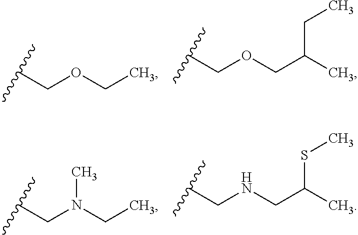

The term “heteroalkyl” preceded by a separate heteroatom refers to a heteroalkyl group bonded through the specified heteroatom. By way of example, a OC₁₋₆heteroalkyl group includes, but is not limited to, the following structures:

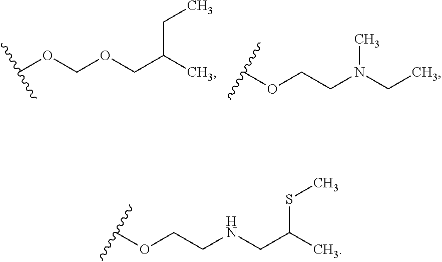

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl (each of which parent groups as defined herein).

The term “alkoxy” refers to an alkyl group, as defined herein, appended to the parent molecular moiety through an oxygen atom.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continues to designate the number of carbon atoms in the aryl ring system. Unless otherwise specified, each instance of an aryl group is independently unsubstituted (an “unsubstituted aryl”) or substituted (a “substituted aryl”) with one or more substituents.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups, wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like), the point of attachment can be on either ring, i.e., either the ring bearing a heteroatom (e.g., 2-indolyl) or the ring that does not contain a heteroatom (e.g., 5-indolyl).

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H^{~}1,5,2-dithiazinyl, dihydrofuro[2,3 b]tetrahydrofuran, furanyl, furazanyl, imidazolidinyl, imidazolinyl, imidazolyl, IH-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

Unless specified to the contrary, the alkyl, alkenyl, alkynyl, heteroalkyl, heteroalkenyl, heteroalkynyl, carbocyclyl, heterocyclyl, aryl, and heteroaryl groups defined herein (and the “ene” versions of said groups) may be substituted or unsubstituted. A substituted group includes a non-hydrogen substituent at a position where, in the unsubstituted version, a hydrogen atom would be found. Substituents include, but are not limited to, halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{a}R^{b}, —NR^{a}C(═O)R^{b}, —NR^{a}C(═O)NR^{a}NR^{b}, —NR^{a}C(═O)OR^{b}, —NR^{a}SO₂Rb, —C(═O)R^{a}, —C(═O)OR^{a}, —C(═O)NR^{a}R^{b}, —OC(═O)NR^{a}R^{b}, —OR^{a}, —SR^{a}, —SOR^{a}, —S(═O)₂R^{a}, —OS(═O)₂R^{a} and —S(═O)₂OR^{a}. R^{a} and R^{b} in this context can be the same or different and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

As used herein, the designation of a polyvalent moiety without specifying the specific order of attachment is intended to cover all possible arrangements. By way of example, a compound represented by the formula:

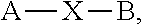

wherein X is NHC(═O) embraces both:

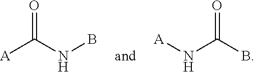

As used herein, a chemical bond depicted: [symbol] represents either a single, double, or triple bond, valency permitting. By way of example,

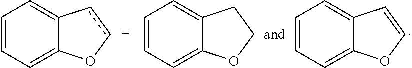

Some compounds disclosed herein may exist as one or more tautomers. Tautomers are interconvertible structural isomers that differ in the position of one or more protons or other labile atoms. By way of example:

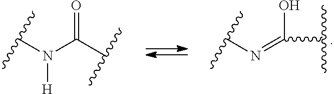

Unless stated to the contrary, a substituent drawn without explicitly specifying the point of attachment indicates that the substituent may be attached at any possible atom. For example, in a benzofuran depicted:

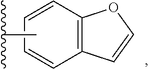

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃. A group having the subscript ‘0’ is understood to represent a null group as well. By way of example, in the compound CH₃—(X)_{z}—CH₃, if X is CH₂ and z is 0, then the compound has the formula CH₃—CH₃.

A bracketed functional group with a subscripted variable should be understood to denote the number of repeated bracketed groups present. For example, a number that is selected from 0 or 1 should be interpreted as follows:

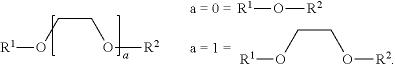

In certain instances, two or more variable groups may together form a ring. It is understood that any depicted atoms separated from the identified groups will themselves form part of the ring:

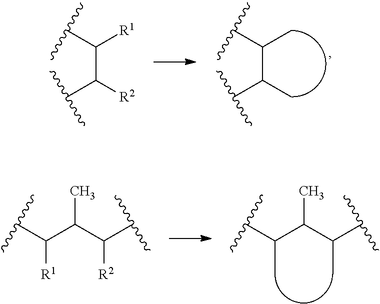

When the variable groups are substituted on an aromatic system, the new ring will be a fused ring, and unless specified to the contrary, may be either aromatic or non-aromatic, carbocyclic or heterocyclic:

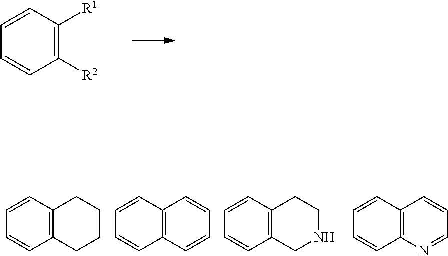

The ring may further be defined by the number of carbon atoms in the specific ring formed by the variable groups, which includes the atoms separating the variable groups:

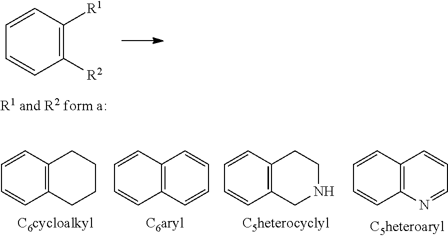

Each of the above results occurs when R¹ and R² together form a six-membered (or six-atom) ring. Other rings, including 3, 4, 5, 7, and 8-member rings, may also be formed, and may be further limited by a specified number of carbon atoms. Although the singular “a ring” may be used to define the group, unless specified to the contrary, both monocyclic and polycyclic rings are possible:

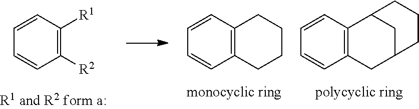

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

As used herein, the chemical group “D” refers to deuterium at an isotopic abundance greater than 25%, 35%, 50%, 60%, 70%, 80%, or 90%. In certain implementations, the chemical group “D” refers to deuterium at an isotopic abundance greater than 50%.

Disclosed herein is a method of treating head and neck cancer in a subject in need thereof, comprising administering to the subject a compound having the formula:

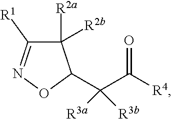

or a pharmaceutically acceptable salt thereof,
wherein:
R¹ is aryl or heteroaryl;
R^{2a} is H or C₁₋₆alkyl, optionally substituted one or more times by halo, OH, OC₁₋₃alkyl, or OC₁₋₃haloalkyl;
R^{2b} is H or C₁₋₆alkyl, optionally substituted one or more times by halo, OH, OC₁₋₃alkyl, or OC₁₋₃haloalkyl; or
wherein R^{2a} and R^{2b}, along with the carbon to which they are attached, may form a C₃₋₈cycloalkyl or C₃₋₈ heterocyclyl;
R^{3a} is H or C₁₋₆alkyl, optionally substituted one or more times by halo, OH, OC₁₋₃alkyl, or OC₁₋₃haloalkyl;
R^{3b} is H or C₁₋₆alkyl, optionally substituted one or more times by halo, OH, OC₁₋₃alkyl, or OC₁₋₃haloalkyl; or
wherein R^{3a} and R^{3b}, along with the carbon to which they are attached, may form a C₃₋₈cycloalkyl or C₃₋₈heterocyclyl; and
R⁴ is C₃₋₈cycloalkyl or C₃₋₈heteorcyclyl.

In some implementations, the compound has the formula:

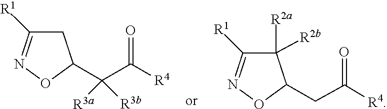

In some implementations, the compound has the formula:

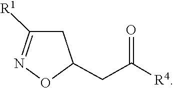

In some implementations, the compound has the formula:

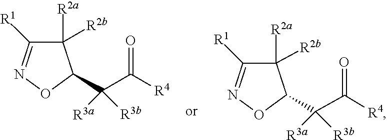

wherein the C-5 carbon on the isoxazole ring has a diastereomeric purity of at least 90%.

In some implementations, R¹ has the formula:

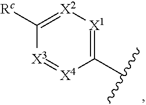

wherein
X¹ is N or CR^{a}, wherein R^{a} is H, F, Cl, Br, C₁₋₃alkyl, OC₁₋₃alkyl, OH, OC₁₋₃alkyl, or OC₁₋₃haloalkyl;
X² is N or CR^{b}, wherein R^{b} is H, F, Cl, Br, C₁₋₃alkyl, OC₁₋₃alkyl, OH, OC₁₋₃alkyl, or OC₁₋₃haloalkyl;
X³ is N or CR^{d}, wherein R^{c} is H, F, Cl, Br, C₁₋₃alkyl, OC₁₋₃alkyl, OH, OC₁₋₃alkyl, or OC₁₋₃haloalkyl;
X⁴ is N or CR^{e}, wherein R^{a} is H, F, Cl, Br, C₁₋₃alkyl, OC₁₋₃alkyl, OH, OC₁₋₃alkyl, or OC₁₋₃haloalkyl; and
R^{c} is H, F, Cl, Br, C₁₋₃alkyl, OC₁₋₃alkyl, OH, OC₁₋₃alkyl, or OC₁₋₃haloalkyl;
wherein no more than one of R^{a}, R^{b}, R^{c}, R^{d}, or R^{e} is OH; and
no more than two of X¹, X², X³, and X⁴ are N.

In some implementations, R¹ is:

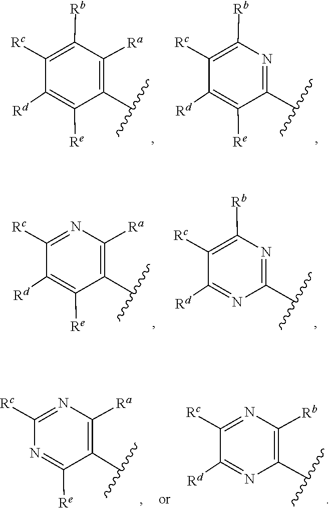

In some implementations, R^{a} is H, F, Cl, CH₃, OH, or OCH₃;

In some implementations, R^{b} is H, F, Cl, CH₃, OH, or OCH₃;

In some implementations, R^{e} is H, F, Cl, CH₃, OH, or OCH₃;

In some implementations, R^{d} is H, F, Cl, CH₃, OH, or OCH₃;

In some implementations, R^{e} is H, F, Cl, CH₃, OH, or OCH₃;

In some implementations, two of R^{a}, R^{b}, R^{c}, R^{d}, and R^{e} are F, and the rest are H.

In some implementations, R^{e} and R^{c} are F, and the rest are H.

In some implementations, R¹ is:

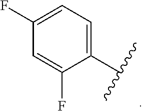 [SMILES: *c1ccc(F)cc1F]

In some implementations, R⁴ is:

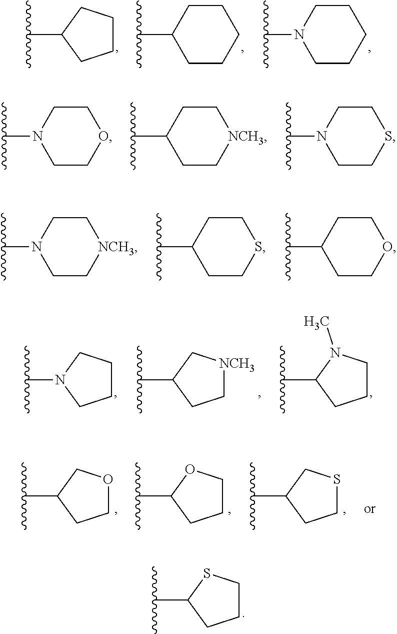

In some implementations, R⁴ is:

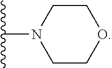 [SMILES: *N1CCOCC1]

In some implementations, the compound has the formula:

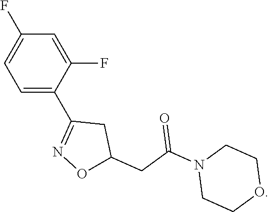 [SMILES: O=C(CC1CC(c2ccc(F)cc2F)=NO1)N1CCOCC1]

In some implementations, the head and neck cancer comprises squamous cell carcinoma of the head and neck.

In some implementations, the head and neck cancer comprises paranasal sinus cancer, nasopharyngeal cancer, hypopharyngeal cancer, oral cancer, oropharyngeal cancer, salivary gland cancer, nasal cavity cancer, or laryngeal cancer.

In some implementations, the head and neck cancer is recurrent head and neck cancer.

In some implementations, the head and neck cancer is metastatic head and neck cancer.

In some implementations, the head and neck cancer is recurrent and metastatic head and neck cancer.

In some implementations, the head and neck cancer comprises stage I head and neck cancer, stage II head and neck cancer, stage III head and neck cancer, or stage IV head and neck cancer.

In some implementations, the subject has previously been administered a different treatment for head and neck cancer.

In some implementations, the subject has previously been administered radiation therapy for head and neck cancer.

In some implementations, the subject has previously been administered chemotherapy for head and neck cancer.

In some implementations, the head and neck cancer is resistant to one or more chemotherapies.

In some implementations, the subject is 65 years of age or older, 19-64 years of age, or 18 years of age or younger.

In some implementations, the subject is male or female.

In some implementations, the compound can be formulated as a pharmaceutical composition for administration. In some implementations, the compound is administered orally. In some implementations, the compound is administered subcutaneously. In some implementations, the compound is administered intravenously.

In some implementations the compounds is administered at a dosage of 1-1,000 mg/day, 1-100 mg/day, 1-10 mg/day, 10-100 mg/day, 25-100 mg/day, 25-250 mg/day, 50-250 mg/day, 50-500 mg/day, 100-500 mg/day, 250-1,000 mg/day, or 500-1,000 mg/day.

In some implementations, the method includes administering at least one other therapy to the subject.

In some implementations, the other therapy comprises surgical removal of a tumor or cancer cells, surgical removal of lymph nodes, radiation therapy, administration of an additional therapeutic agent, or a combination thereof.

In some implementations, the additional therapeutic agent comprises a PD-1 or PD-L1 inhibitor.

In some implementations, the PD-1 or PD-L1 inhibitor comprises acrixolimab, pembrolizumab, cemiplimab, cosibelimab, atezolizumab, avelumab, durvalumab, nivolumab, dostarlimab, retifanlimab, toripalimab, vopratelimab, spartalizumab, camrelizumab, sintilimab, tislelizumab, INCMGA00012, AMP-224, AMP-514, KN035, AUNP12, CA-170, or BMS-986189.

In some implementations, the additional therapeutic agent comprises an EGFR inhibitor, a monoclonal antibody, a platin, or a taxoid.

In some implementations, the additional therapeutic agent comprises bleomycin sulfate, cetuximab, docetaxel, hydroxyurea, pembrolizumab, cemiplimab, atezolizumab, avelumab, durvalumab, toripalimab-tpzi, methotrexate sodium, nivolumab, erlotinib, vandetanib, gefitinib, lapatinib, larotrectinib, entrectinib, carboplatin, cis-platin, paclitaxel, fluorouracil, or a combination thereof.

### EXAMPLES

The following examples are for the purpose of illustration of the invention only and are not intended to limit the scope of the present invention in any manner whatsoever.

CPSI 1306 was generously provided by L2 Diagnostics, LLC, New Haven, CT. HNSCC cell lines CAL-27 SCC-83 and CA-83, were cultured in DMEM, 10% FBS, and 1% Pen-strep glutamine with supplemental 1% non-essential amino acids. MOC2 cells were cultured in IMDM/F12, 2:1, with 5% FBS, 1% Pen-strep glutamine, 5 ug/mL insulin, 40 ng/ml hydrocortisone, and 5 ng/ml human recombinant EGF.

Cell viability assay was performed using alamarBlue dye (Thermo Fisher Scientific). 2×10³ cells were seeded per well in 100 ul of medium into 96-well plates. Cells were allowed to incubate overnight and serially treated with 100 μM with CPSI-1306 for 72 hours. After incubation, 10 ul alamarBlue dye was added and incubated for 6 hours. Absorbance was measured at 570 nm and 600 nm, and percent viability was calculated as described.

Tumors were formalin-fixed and paraffin-embedded as described in Front Immunol 2022; 13:932742. For immunohistochemistry (IHC), Tissue sections (5 μm) were stained for MIF (Proteintech, 20415-1-AP) with secondary biotinylated antibody for goat anti-rabbit (Vector Labs, BA-1000) along with a hematoxylin counterstain. For immunofluorescence (IF), tissue sections (5 μm) were stained for CD8 (Invitrogen, A21434) and Granzyme B (Abcam, AB4059) with the secondary antibodies Alexa Fluor 488-conjugated goat anti-rabbit (Invitrogen, A11034) and Alexa Fluor 555 goat anti-rat (Invitrogen, A21434). Each section was then counterstained with DAPI (BioLegend, San Diego, CA). Confocal imaging was performed using a Zeiss LSM 700 confocal microscope (CarlZeiss, Munich, Germany). Regions of positive stains were quantified using ImageJ for both IHC and IF.

Human and mouse primer sequences for BCL2, VEGFA, EGFR, and CCND1 were obtained using PRIMER BANK (http://pga.mgh.harvard.edu/primerbank/index.html). PCR amplification was performed using the PowerUp SYBR Green Master Mix (Thermofisher Scientific, Foster Coty, CA, USA) for detection. Data normalization was performed to housekeeping genes GAPDH and β-actin.

Proteins were extracted from in vitro cultures of CAL27, SCC83, CA83, and MOC2 treated with CPSI-1306 at 1 and 10 UM concentrations for 24 h. 30 μg of protein was loaded onto a 10% Tris-HCL gel. Proteins were transferred to a PVDF membrane, blocked using 5% non-fat dry milk, and incubated with the rabbit phospho-p44/42 MAPK (Erk1/2) (Cell Signaling, 4370S), p44/42 MAPK (Erk1/2) (Cell Signaling, 4695S), and Rabbit GAPDH (Cell Signaling, 2118S) primary antibody overnight. Blots were incubated with goat anti-rabbit HRP linked secondary antibody (Thermo Fisher Scientific, Rockland, IL). Chemiluminescence was detected by ECL western blotting substrate (Thermo Scientific, Waltman, MA). Quantification of images was performed using the ImageJ FIJI package (Ver. 2.1.0) in ImageJ.

Experiments were approved by the Institutional Animal Care and Use Committee of the Ohio State University (IACUC), and animals were housed and cared for by University Laboratory Animal Resources guidelines. Female C57BL/6 mice were purchased from Jackson Laboratory (Bar Harbor, ME) and were injected orthotopically with MOC2 (3×10⁴ cells) into the right buccal cavity. Mice were randomly assigned to vehicle control (n=10) and CPSI-1306 (n=10) groups after injection. On day 5 post-injection, mice began treatment via oral gavage with CPSI-1306 (20 mg/kg in 1.5% DMSO, 0.5% methylcellulose) or vehicle for 2 weeks, 5 times/week. Mice weight and tumor volume measurements were recorded twice a week. Tumors were analyzed for volume via the equation V=L×S²×0.5, where L is the longest diameter, and S is the short diameter. At terminal sacrifice, spleen, tumors, and lymph nodes were harvested for flow cytometry and gene expression analysis. Tumors were imaged and analyzed for volume at sacrifice.

For in vitro analysis of MIF expression, TE1177, SCC83, CA83, and CAL27 cells were incubated with MIF conjugated antibody (Invitrogen, 367401) and MOC2 cells were incubated with MIF fluorochrome conjugated antibody (ProteinTech, 20415-1-AP). Single cell suspensions were generated from spleens, draining lymph nodes, and tumors of experimental mice, which were stained extracellularly with fluorochrome conjugated antibodies for CD4, CD8, CD45, CTLA4, TIM3, TIGIT, LAG3, CD69, and PD1 (BioLegend. San Jose, CA, USA). Cells were also incubated with antibodies targeting IL-2, II-10, IFN-γ, Perforin, Granzyme B, and TNF-α to identify intracellularly expressed markers (BioLegend. San Jose, CA, USA). For myeloid subsets, cells were incubated with fluorochrome conjugated antibodies for Cd11b, CD11c, IA/IE, CD80, F4/80, Ly6C, Ly6G, and PD-L1 (BioLegend. San Jose, CA, USA). Samples were analyzed using FACS Celesta flow cytometer (BD Biosciences, San Jose, CA). Flow cytometric analysis was completed using FlowJo (Tree Star Inc., Ashland, OR, USA).

Statistical analyses were conducted using GraphPad Prism software v9.2.0 (GraphPad Software, San Diego, CA, USA). Student's t test (two-sided) was used to determine the statistical significance between the groups.

### Example 1: CPSI-1306 Reduces Tumor Progression in an Orthotopic In Vivo Model of HNSCC

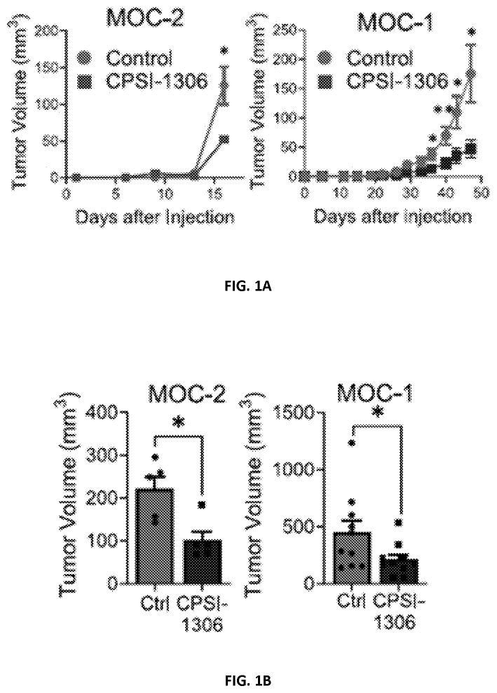

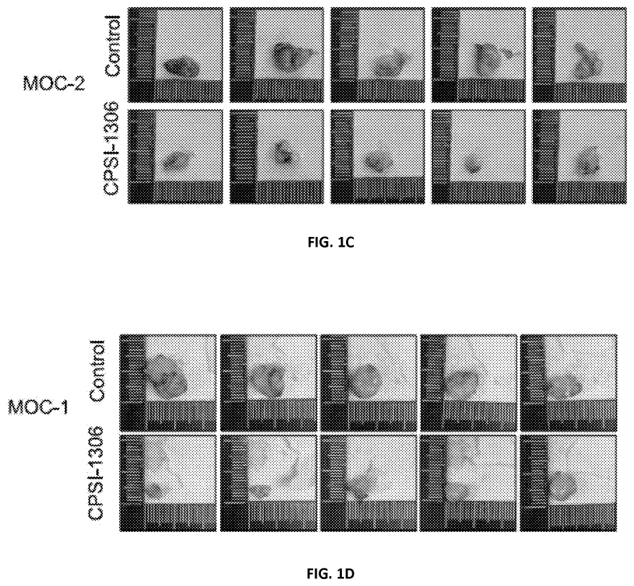

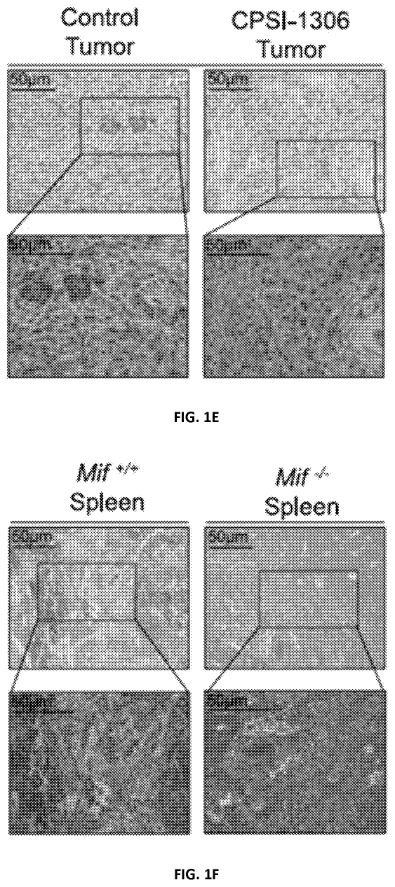

We determined the therapeutic potential of CPSI-1306 in an orthotopic model of HNSCC in vivo using the well characterized MOC-2 HNSCC cell line. MOC-2 tumor bearing mice were treated with either ibrutinib (20 mg/kg/day for 2 weeks) or vehicle via oral gavage, 5 days following tumor injections. Mouse tumors were measured, with reduced tumor volumes noted at Day 17 post injection (FIG. 1A). Mouse weights were recorded, with no significant weight differences but on average being higher with CPSI-1306 treatment (FIG. 1B). Mouse tumors were also measured and quantified using ImageJ post sacrifice, with volumes similarly being significantly reduced with treatment (FIGS. 1C-D). This data suggests that CPSI-1306 can reduce head and neck cancer cell growth in vivo. We further characterized expression of MIF in mice bearing tumors after injection with MOC2 HNSCC cells. As expected, MOC2 tumor bearing mice expressed high MIF levels, in tumors of both CPSI-1306 and vehicle treated mice (FIG. 1E). MIF expression was also detected in spleens of tumor bearing mice, but not on MIF knock out mice (FIG. 1F).

### Example 2: CPSI-1306 Enhances T-Cell Infiltration to the HNSCC Tumor Microenvironment

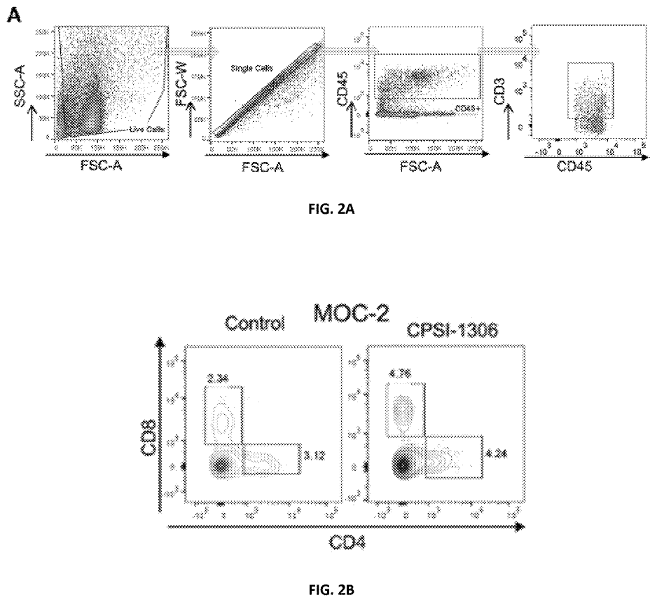

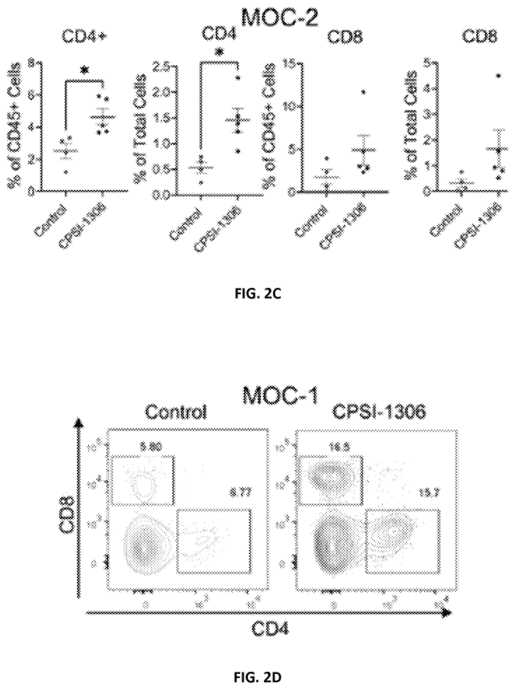

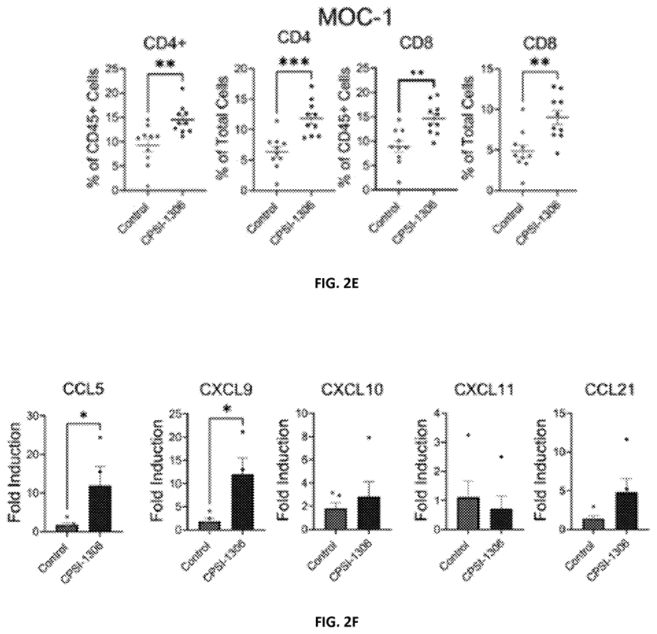

We determined potential mechanisms underlying CPSI-1306 mediated anticancer effects. First, we analyzed CD4+ and CD8+ T cell infiltration into the tumor microenvironment of MOC2 tumor bearing mice (FIG. 2A). CD4+ T-cell infiltration was significantly increased in tumors of CPSI treated mice compared to vehicle controls, while a modest but not significant increase in CD8+ T cells was observed in CPSI-1306 treated tumor-bearing mice (FIG. 2B). These findings correspond to previous studies showing that MIF inhibition may promote anticancer effects through improved immune activation and function. To determine potential mechanisms underlying the increased T cell infiltration of CPSI-1306 treated tumor bearing mice, we analyzed gene expression levels of chemokines associated with enhanced T cell recruitment. We observed increased expression levels of CCL5 and CXCL9 in the tumor microenvironment of CPSI-1306 treated tumor bearing mice compared to vehicle control group (FIG. 2C). Taken together, our data indicates that reduction in tumor progression in CPSI-treated mice is associated with the upregulation of antitumoral T cell attracting chemokines and increased recruitment of T cells to the tumor microenvironment.

### Example 2: CPSI-1306 Enhances T-Cell Infiltration to the HNSCC Tumor Microenvironment

We determined potential mechanisms underlying CPSI-1306 mediated anticancer effects. First, we analyzed CD4+ and CD8+ T cell infiltration into the tumor microenvironment of MOC2 tumor bearing mice (FIG. 2A). CD4+ T-cell infiltration was significantly increased in tumors of CPSI treated mice compared to vehicle controls, while a modest but not significant increase in CD8⁺ T cells was observed in CPSI-1306 treated tumor-bearing mice (FIG. 2B). These findings correspond to previous studies showing that MIF inhibition may promote anticancer effects through improved immune activation and function [29]. To determine potential mechanisms underlying the increased T cell infiltration of CPSI-1306 treated tumor bearing mice, we analyzed gene expression levels of chemokines associated with enhanced T cell recruitment. We observed increased expression levels of CCL5 and CXCL9 in the tumor microenvironment of CPSI-1306 treated tumor bearing mice compared to vehicle control group (FIG. 2C). Taken together, our data indicate that reduction in tumor progression in CPSI-treated mice is associated with the upregulation of antitumoral T cell attracting chemokines and increased recruitment of T cells to the tumor microenvironment.

### Example 3: CPSI-1306 Completely Abrogates Immunosuppressive Checkpoint Markers TIGIT, TIM3, and CTLA-4, but not PD-1 on Tumor Infiltrating CD8+ T Cells

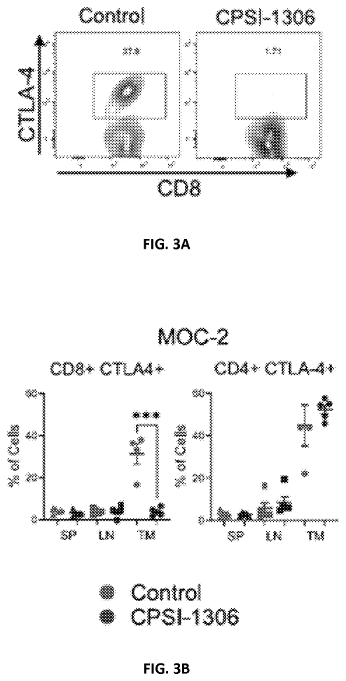

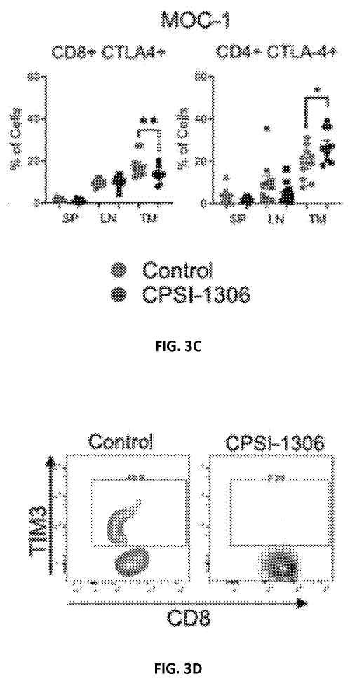

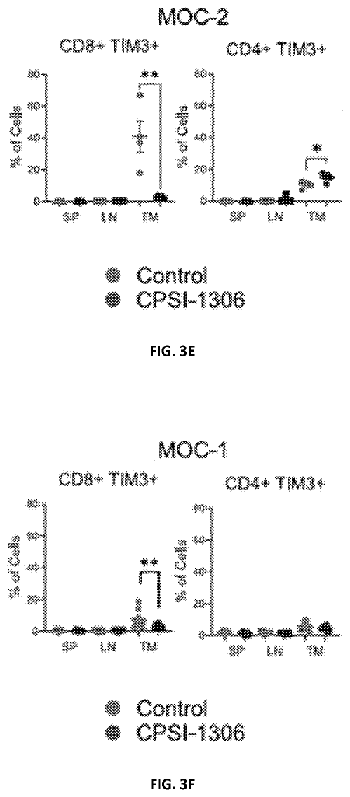

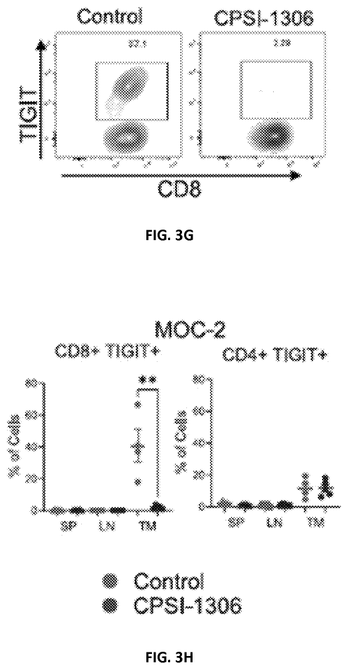

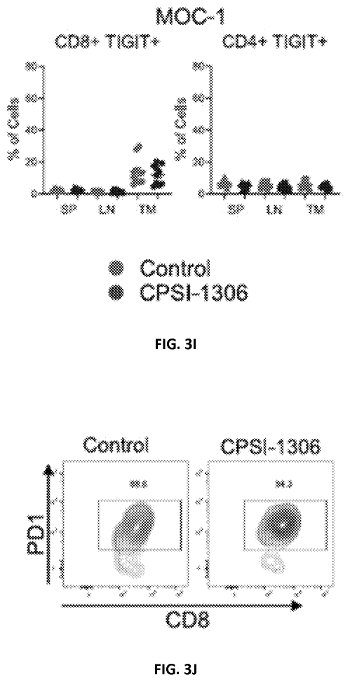

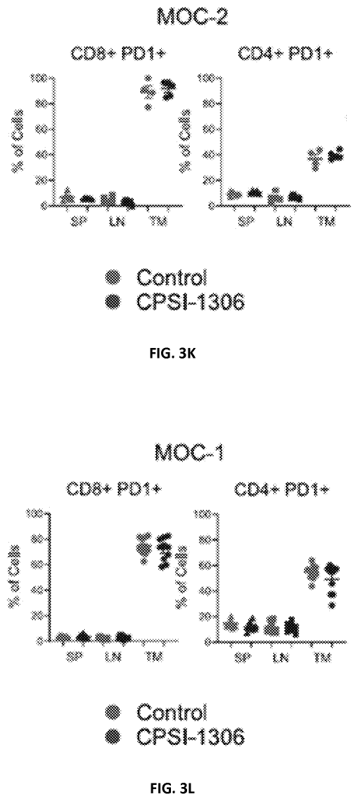

We analyzed functional states of T cell subsets recruited to the HNSCC tumor microenvironment following CPSI-1306 treatment. In HNSCC, checkpoint inhibitor markers TIM-3, CTLA-4, TIGIT, and PD-1 are overexpressed in T cells and are linked to T-cell exhaustion and dysfunction, dampening effector T-cell responses. Our analysis of tumor infiltrating T cells revealed that CPSI-1306 treatment completely abrogated expression of CTLA-4, TIM3 and TIGIT in CD8+ tumor infiltrating lymphocytes compared to vehicle controls, with no significant changes in the spleens or draining lymph nodes (FIGS. 3A-3C). No significant change in PD1 expression was noted in CD8+ T cells in the spleen, draining lymph nodes, or tumors (FIG. 3D). We also analyzed expression of these immunosuppressive markers on CD4+ T cells in the spleen, lymph node, and tumors of mice treated with vehicle and CPSI-1306. While these markers were significantly increased in tumor infiltrating CD4+ T cells, compared to spleens and draining lymph nodes, CPSI-1306 treatment did not reduce expression of these checkpoint markers CTLA-4, TIM3, TIGIT, and PD1 (FIG. 3E-3H). Our data suggests that CPSI-1306 selectively targets tumor infiltrating CD8+ T cells to improve anti-tumor immunity in the HNSCC tumor microenvironment.

### Example 4: CPSI-1306 Enhances T Cell Effector Functions in Tumors and Draining Lymph Nodes of HNSCC Tumor-Bearing Mice

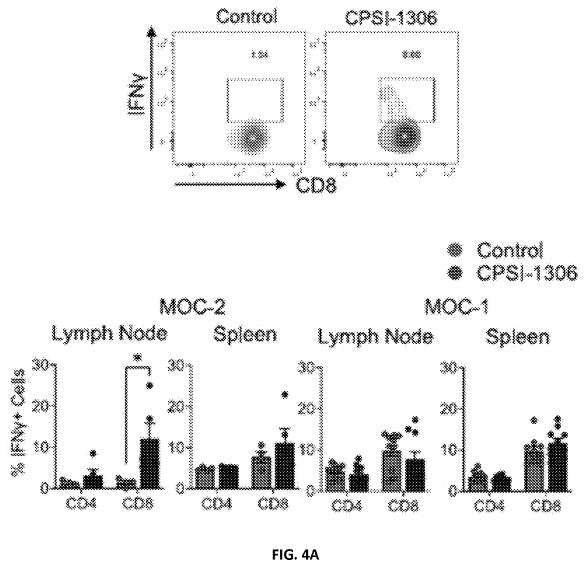

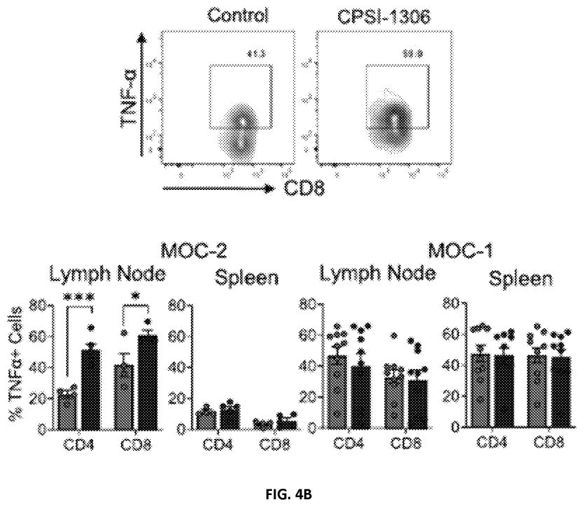

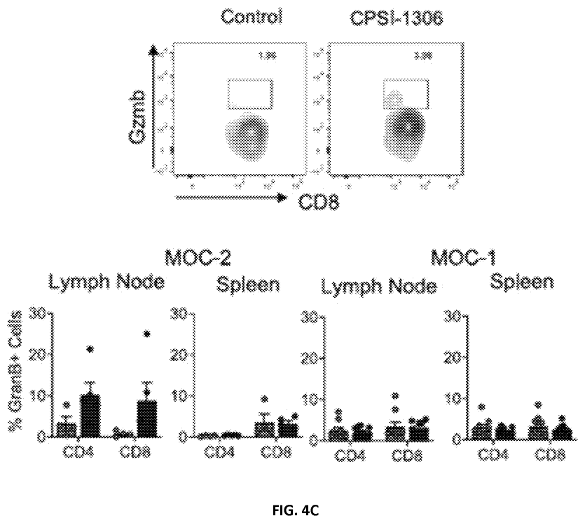

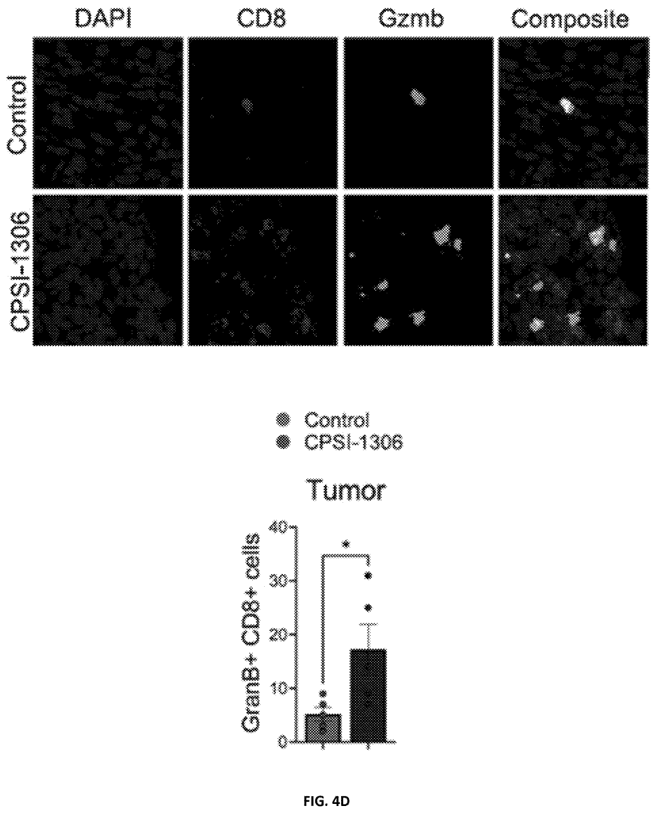

We analyzed the effect of CPSI-1306 treatment on T cell effector function in HNSCC tumor bearing mice. To do this, we examined intracellular expression of IFN-γ, TNFα, and GZMB in CD4+ and CD8+ T cells in spleens, draining lymph nodes, and tumors of tumor bearing mice treated with CPSI-1306 or vehicle control. We observed significantly increased levels of IFN-γ producing CD8+ T cells in draining lymph nodes compared to vehicle control mice (FIG. 4A). Both CD4+ and CD8+ T cells in the lymph nodes displayed increased levels of TNF-α compared to control treated mice (FIG. 4B). Although Gzmb was slightly but not significantly increased in CD4+ and CD8+ T cells in the lymph nodes with CPSI-1306 treatment (FIG. 4C), Gzmb producing CD8+ T cells were significantly increased in the tumor microenvironment of CPSI-1306 treated mice compared to vehicle controls (FIG. 4D), demonstrating higher CD8 cytotoxic activity in the primary tumor. Taken together, in addition to modulating CD8+ tumor cell checkpoint markers, CPSI-1306 may enhance antitumoral immune responses in the primary tumors and draining lymph nodes of mice, suggesting an immunomodulatory mechanism of anticancer activity by the MIF inhibitor CPSI-1306

### Example 5: Effect of CPSI-1306 on MIF Mediated Signaling Pathways on HNSCC Cells

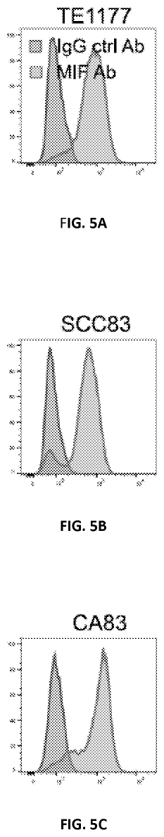

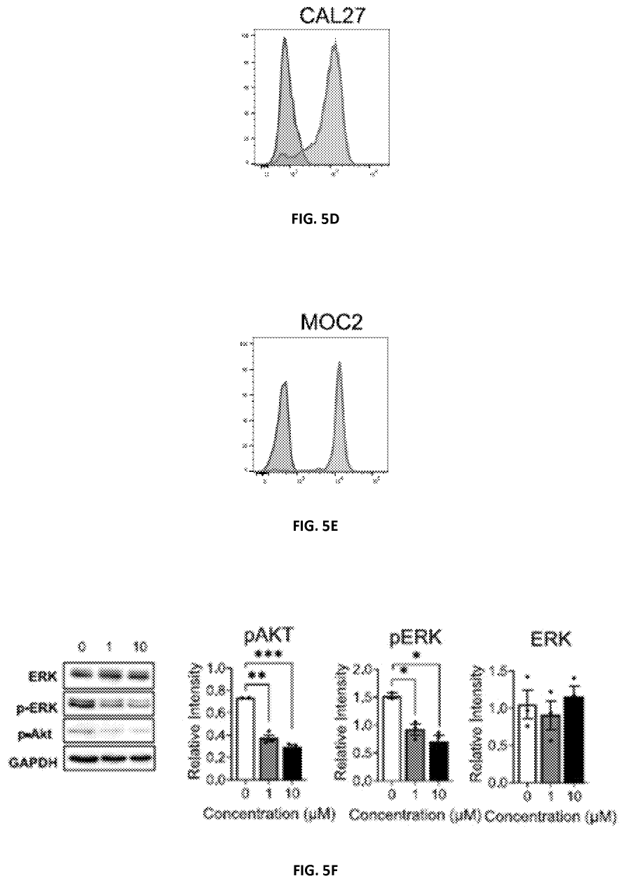

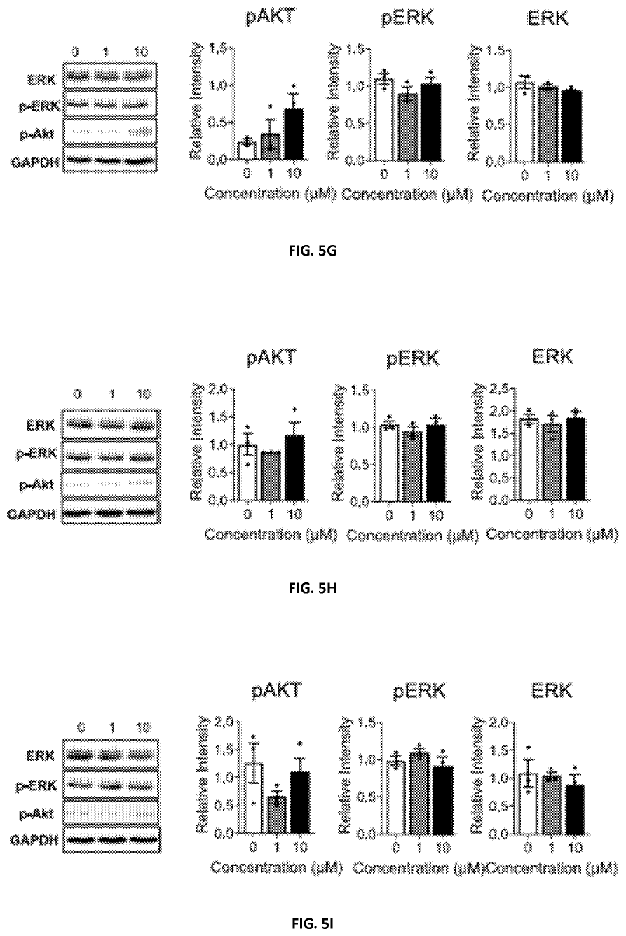

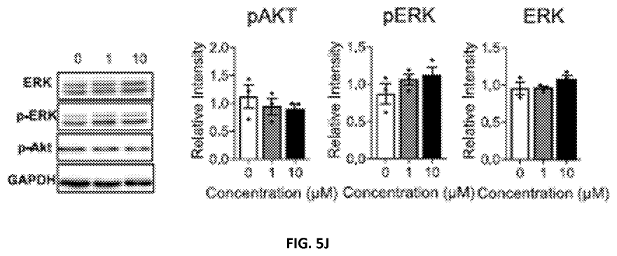

MIF interactions with CD74 leads to activation of the PI3K/AKT and ERK1/2 MAPK pathways, which mediate expression of cancer associated apoptotic and proliferative factors and impact the tumor micro-environment. Given that MIF mediates cancer growth and progression via the PI3K/Akt and ERK1/2 MAPK cascades, we determined the expression of pAKT, ERK, and pERK after treatment with CPSI-1306. We first confirmed expression of MIF in normal (TE1177), and HNSCC (SCC83, CA83, CAL27 and MOC2) cell lines (FIG. 5A-5E). Our analysis for the non-cancer human epithelial cell line TE1177 showed a reduction in pAKT and pERK levels (FIG. 5F). Interestingly, in MOC2, CA83, SCC83, and CAL27 cell lines, we observed that pAKT and pERK were not significantly reduced with treatment of CPSI-1306 at 1 or 10 μM (FIGS. 5G-5J). The data suggest that MIF inhibition via CPSI-1306 may not significantly affect these pathways in vitro in these oral cancer cell lines. Given that other receptor-ligand interactions may exert influence on the PI3K/AKT and ERK1/2 MAPK axis, such as EGFR, our results demonstrate that MIF inhibition with CPSI-1306, may have minimal direct effects on cell signaling pathways associated with MAPK and AKT signaling in HNSCC cells. Further, CPSI-1306 did not inhibit the proliferation of HNSCC cells in vitro. Taken together, our data suggest that MIF inhibition using CPSI-1306 indirectly inhibits cancer progression, by enhancing T cell mediated cytotoxicity of HNSCC cells in the tumor microenvironment.

MIF is a pluripotent cytokine with proinflammatory characteristics that promote HNSCC carcinogenesis. We demonstrated high expression of MIF in HNSCC cell lines, as well as in MOC2 HNSCC in vivo model. Noticeably, the increased expression of MIF in the more aggressive CA83 and MOC2 cancer cell lines suggests a positive correlation between MIF overexpression and increased HNSCC aggressiveness. This agrees with previous studies demonstrating the potential for MIF as a biomarker for HNSCC and target for HNSCC treatment.

Although MIF inhibitors in general have shown positive results in cell-based analysis, our focus on CPSI-1306, a special isooxaline class of inhibitors, was based on its non-toxic and well tolerated pharmacokinetic profile. We did not observe significant anti-proliferative effects of CPSI-1306 on mouse and human HNSCC cells in vitro, which suggested that the antitumoral effects of CPSI-1306 in HNSCC is largely due to enhancement of T cell immune response pathways. Interestingly, while CPSI-1306 did not reduce MOC2 cell proliferation in vitro, we observed that CPSI-1306 significantly reduced MOC2 tumor volumes in vivo. Additionally, tumors were found to be less inflamed and visibly less vascular, suggesting an antitumoral effect on the HNSCC tumor microenvironment.

The absence of an inhibitory effect on pERK and pAKT phosphorylation in human and mouse oral cancer cell lines by CPSI-1306 mediated MIF inhibition, suggests active MIF independent activation of these pathways in HNSCC cancer cells. Interestingly, DDT (MIF 2), another member of the MIF family, is not inhibited by CPSI-1306, and has been shown to promote tumor proliferation and development via similar mechanisms as MIF. As such, dual inhibition of MIF and DDT may prove to be more beneficial than MIF inhibition alone in HNSCC treatment and warrants further exploration. Our results may also suggest a potentially viable strategy via combinatorial approaches with EGFR inhibitors currently used in HNSCC treatment.

A key finding of this study was a complete abrogation of exhaustion markers CTLA-4, TIGIT, and TIM3 in CD8+ T-cells in the tumor microenvironment. In addition to CTLA4, TIGIT, and TIM3 inhibitors are growingly becoming used in clinical studies to target cancer, and our data demonstrates the potential of CPSI-1306 mediated MIF inhibition to target checkpoint markers expressed on T cells. Our results indicate that CPSI-1306 contributes to reducing immunosuppressive markers that downregulate cytotoxic T-cell function, thereby restoring T cell antitumoral properties in HNSCC. Checkpoint inhibition has been a large focus of immunotherapy, and current FDA approved inhibitors target CTLA-4, PD-1, and PD-L1, with PD-1 inhibitors pembrolizumab and nivolumab being approved for HNSCC. These immune checkpoint inhibitors (ICIs) often are minimally effective in patients with “cold” tumors, with only a fraction of HNSCC patients responding to ICIs. However, combination therapies of ICIs with MIF inhibitors in melanoma have demonstrated enhanced reprogramming of innate immunity and increased potentiation of CD8+ T cells, leading to reduced tumor progression. Therefore, given that CPSI-1306 did not affect PD-1 expression on T cells in the tumor microenvironment, a combinatorial approach with CPSI-1306 and PD-1 inhibitor could serve as a potential therapeutic strategy for HNSCC.

As evidenced by increased levels of chemokines CCL5 and CXCL9, linked to Th1 and T effector cell responses, MIF inhibition via CPSI-1306 may contribute to a reprogramming of the tumor microenvironment characterized by increased infiltration of antiumoral T cells to facilitate an improved tumor response. CXCL9, is associated with Th1 responses and correlates with enhanced antitumoral immune cell infiltration. CCL5, is also known to recruit antitumoral T cells to the tumor microenvironment, thereby enhancing the immunotherapy response in various cancers. IFNγ is linked to immune activation when secreted by T cells, and promotes production of Gzmb by CTLs leading to apoptosis in cancer cells. Therefore, our observed increase in CD8+ Gzmb producing cells within the tumor of CPSI-1306 treated mice in our study suggests a more robust and efficient antitumoral immunity against HNSCC, in response to CPSI-1306 treatment.

In conclusion, our findings suggest for the first time that CPSI-1306 can reverse immunosuppressive T cell phenotypes in the tumor microenvironment in an in vivo murine model of HNSCC. CPSI-1306 mediated MIF inhibition led to significant antitumor response in HNSCC, due to heightened immune cell filtration coupled with enhanced T cell antitumoral activity, and is associated with reduced tumor growth in vivo. Targeting MIF via the small-molecule inhibitor CPSI-1306 is a potent strategy against HNSCC, which currently lacks formidable and effective targeted therapy options, and could especially be a promising therapeutic in combination with existing therapies.

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less; however, other combinations of steps, elements, components, and constituents are included, even though not explicitly stated. The term “comprising” and variations thereof, as used herein, are used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method of treating head and neck cancer in a subject in need thereof, comprising administering to the subject a compound having the formula: 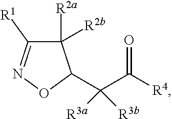
or a pharmaceutically acceptable salt thereof,
wherein:
R¹ is aryl or heteroaryl;
R^{2a} is H or C₁₋₆alkyl, optionally substituted one or more times by halo, OH, OC₁₋₃alkyl, or OC₁₋₃haloalkyl;
R^{2b} is H or C₁₋₆alkyl, optionally substituted one or more times by halo, OH, OC₁₋₃alkyl, or OC₁₋₃haloalkyl; or
wherein R^{2a} and R^{2b}, along with the carbon to which they are attached, form a C₃₋₈cycloalkyl or C₃₋₈heterocyclyl;
R^{3a} is H or C₁₋₆alkyl, optionally substituted one or more times by halo, OH, OC₁₋₃alkyl, or OC₁₋₃haloalkyl;
R^{3b} is H or C₁₋₆alkyl, optionally substituted one or more times by halo, OH, OC₁₋₃alkyl, or OC₁₋₃haloalkyl; or
wherein R^{3a} and R^{3b}, along with the carbon to which they are attached, form a C₃₋₈cycloalkyl or C₃₋₈heterocyclyl; and
R⁴ is C₃₋₈cycloalkyl or C₃₋₈heteorcyclyl.

2. The method of claim 1, wherein the compound has the formula:

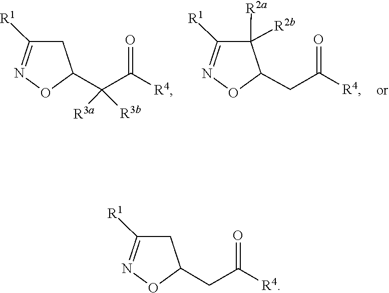

3. The method of claim 1, wherein the compound has the formula: 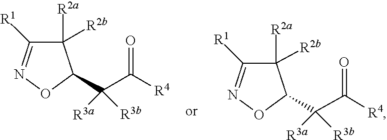
wherein the C-5 carbon on the isoxazole ring has a diastereomeric purity of at least 90%.

4. The method of claim 1, wherein R¹ has the formula: 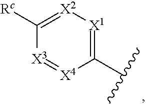
wherein
X¹ is N or CR^{a}, wherein R^{a} is H, F, Cl, Br, C₁₋₃alkyl, OC₁₋₃alkyl, OH, OC₁₋₃alkyl, or OC₁₋₃haloalkyl;
X² is N or CR^{b}, wherein R^{b} is H, F, Cl, Br, C₁₋₃alkyl, OC₁₋₃alkyl, OH, OC₁₋₃alkyl, or OC₁₋₃haloalkyl;
X³ is N or CR^{a}, wherein R^{e} is H, F, Cl, Br, C₁₋₃alkyl, OC₁₋₃alkyl, OH, OC₁₋₃alkyl, or OC₁₋₃haloalkyl;
X⁴ is N or CR^{e}, wherein R^{a} is H, F, Cl, Br, C₁₋₃alkyl, OC₁₋₃alkyl, OH, OC₁₋₃alkyl, or OC₁₋₃haloalkyl; and
R^{c} is H, F, Cl, Br, C₁₋₃alkyl, OC₁₋₃alkyl, OH, OC₁₋₃alkyl, or OC₁₋₃haloalkyl;
wherein no more than one of R^{a}, R^{b}, R^{c}, R^{d}, or R^{e} is OH; and
no more than two of X¹, X², X³, and X⁴ are N.

5. The method of claim 1, wherein R¹ is:

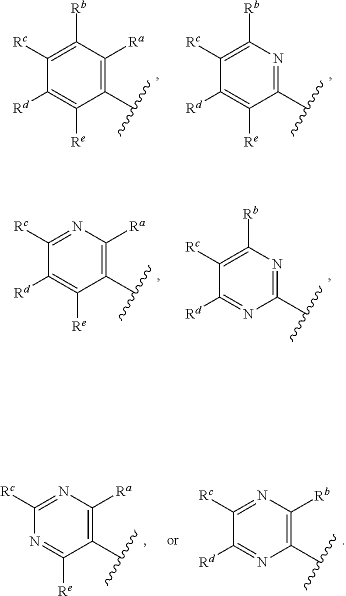

6. The method of claim 4, wherein
R^{a} is H, F, Cl, CH₃, OH, or OCH₃;
R^{b} is H, F, Cl, CH₃, OH, or OCH₃;
R^{c} is H, F, Cl, CH₃, OH, or OCH₃;
R^{d} is H, F, Cl, CH₃, OH, or OCH₃; and
R^{e} is H, F, Cl, CH₃, OH, or OCH₃;

7. The method of claim 4, wherein two of R^{a}, R^{b}, R^{c}, R^{d}, and R^{e} are F, and the rest are H.

8. The method of claim 4, wherein R^{e} and R^{c} are F, and the rest are H.

9. The method of claim 1, wherein R¹ is:

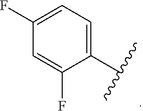 [SMILES: *c1ccc(F)cc1F]

10. The method of claim 1, wherein R⁴ is:

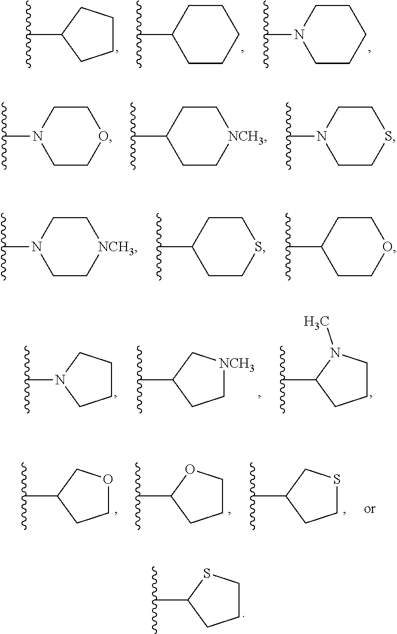

11. The method of claim 1, wherein the compound has the formula:

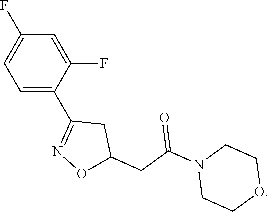 [SMILES: O=C(CC1CC(c2ccc(F)cc2F)=NO1)N1CCOCC1]

12. The method of claim 1, wherein the head and neck cancer comprises squamous cell carcinoma of the head and neck.

13. The method of claim 1, wherein the head and neck cancer comprises paranasal sinus cancer, nasopharyngeal cancer, hypopharyngeal cancer, oral cancer, oropharyngeal cancer, salivary gland cancer, nasal cavity cancer, or laryngeal cancer.

14. The method of claim 1, wherein the head and neck cancer is recurrent head and neck cancer, metastatic head and neck cancer, or recurrent and metastatic head and neck cancer.

15. The method of claim 1, wherein the subject has previously been administered a different treatment for head and neck cancer.

16. The method of claim 1, wherein the subject has previously been administered radiation therapy or chemotherapy for head and neck cancer.

17. The method of claim 1, wherein the head and neck cancer is resistant to one or more chemotherapies.

18. The method of claim 1, further comprising administering at least one other therapy to the subject.

19. The method of claim 18, wherein the other therapy comprises surgical removal of a tumor or cancer cells, surgical removal of lymph nodes, radiation therapy, administration of an additional therapeutic agent, or a combination thereof.

20. The method of claim 19, wherein the additional therapeutic agent comprises a PD-1 or PD-L1 inhibitor, an EGFR inhibitor, a monoclonal antibody, a platin, or a taxoid.
